(19) United States
(12) Patent Application Publication
(10) Pub. No.: US 2026/0325839 A1
(43) Pub. Date: Oct. 22, 2026

(54) CHARACTERIZATION OF ELECTRICAL STIMULATION PARAMETERS

(71) Applicant: Noctrix Health, Inc., Pleasanton, CA (US)

(72) Inventors: Shriram Raghunathan, Castro Valley, CA (US); Andrew Witte, Palo Alto, CA (US)

(21) Appl. No.: 19/562,481

(22) Filed: Mar. 10, 2026

Related U.S. Application Data
(63) Continuation of application No. 19/352,034, filed on Oct. 7, 2025.
Application No. 19/352,034 is a continuation of application No. PCT/US2024/024116, filed on Apr. 11, 2024.
(60) Provisional application No. 63/458,485, filed on Apr. 11, 2023.
(60) Provisional application No. 63/497,667, filed on Apr. 21, 2023.

(51) Int. Cl. A61N 1/36; A61N 1/04

(52) U.S. Cl. CPC A61N 1/36003; A61N 1/0456; A61N 1/36034

(57) ABSTRACT

An electrostimulation electronics unit can include control waveform generation circuitry that provides at least one alternating current (AC) first control waveform. A first amplifier can be included, to supply a controlled current first electrostimulation output waveform via a first patient electrode terminal. Additionally, a second amplifier with an input coupled to an output of the first amplifier can be included to supply a controlled voltage second electrostimulation output waveform via a second patient electrode terminal. The first and second electrostimulation output waveforms are operable together to provide charge balanced electrostimulation to the patient over a full AC electrostimulation cycle.

### PRIORITY

This application is a continuation of U.S. patent application Ser. No. 19/352,034, filed Oct. 7, 2025, which is a continuation from International Application No. PCT/US2024/024116, filed Apr. 11, 2024, and published as WO 2024/215916 on Oct. 17, 2024, which claims the benefit of priority to U.S. Provisional Application Ser. No. 63/458,485 filed Apr. 11, 2023, and U.S. Provisional Application Ser. No. 63/497,667 filed Apr. 21, 2023, all of which are incorporated by reference herein in their entirety.

### BACKGROUND

Electrical nerve stimulation can be used to treat one or more conditions, such as chronic or acute pain, epilepsy, depression, bladder disorders, or inflammatory disorders. Certain neurological disorders can be attributed to overactivity of sensory or other peripheral nerve fibers which can disrupt quality of life, and/or the processing of such neural activity in the brain. Restless Legs Syndrome (RLS) and Periodic Limb/Leg Movement Disorder (PLMD) are two such neurological conditions that can significantly affect sleep in human subjects. RLS (which can also be called Willis-Ekbom Disease (WED)) subjects can experience uncomfortable tingling sensations in their lower limbs (legs) and, less frequently in the upper limbs (arms). RLS is characterized by an uncontrollable urge to move the affected limb(s). Such sensations can often be temporarily relieved by moving the limb voluntarily but doing so can interfere with the RLS subject's ability to fall asleep. PLMD subjects can experience spontaneous movements of the lower legs during periods of sleep, which can cause the PLMD subject to wake up. RLS can be a debilitating sleep disorder and can be comorbid with other sleep disorders such as insomnia or sleep apnea syndrome (SAS).

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 depicts a perspective view of a wearable electrostimulation device.

FIG. 2A depicts an example of a wearable electrostimulation device in use on a subject.

FIG. 2B depicts an example of a wearable electrostimulation device in use on a subject.

FIG. 2C depicts an example of a wearable electrostimulation device in use on a subject.

FIG. 3 is a schematic representation of portions of an example of a wearable electrostimulation device.

FIG. 4 is a schematic representation of portions of an example of an electrostimulation electronics unit of a wearable electrostimulation device.

FIG. 5A is a schematic representation of an electrostimulation signal amplification unit of a wearable electrostimulation device.

FIG. 5B depicts an exemplary output waveform from a waveform generator of the exemplary amplification unit of FIG. 5A.

FIG. 5C depicts an exemplary output waveform from a first amplifier A1 of the exemplary amplification unit of FIG. 5A.

FIG. 5D depicts exemplary output waveforms from a first amplifier A1 and a second amplifier A2 of the exemplary amplification unit of FIG. 5A.

FIG. 5E depicts exemplary sensing circuit waveforms from respective boost circuits of a first amplifier A1 and a second amplifier A2 of the exemplary amplification unit of FIG. 5A.

FIG. 6A shows an example of an oscilloscope waveform produced by a wearable electrostimulation device, the waveform involving a rapid change in impedance from about 1 kΩ toward a value indicating an open circuit.

FIG. 6B shows an example of an oscilloscope waveform produced by a wearable electrostimulation device, the waveform involving a rapid change in impedance from a value indicating an open circuit toward a value of about 1 kΩ.

FIG. 6C shows an example of an oscilloscope waveform produced by a wearable electrostimulation device, the waveform involving a rapid change in impedance from a value of about 1 kΩ toward a value of about 100Ω.

FIG. 6D depicts a magnified and stretched view of the example of the oscilloscope waveform of FIG. 6C.

FIG. 6E shows an example of an oscilloscope waveform produced by a wearable electrostimulation device, the waveform involving a rapid change in impedance from a value of about 10002 toward a value of about 1 kΩ.

FIG. 7 is a graph depicting an example of an initial impedance drop exhibited by the first and second skin electrodes at a beginning of an electrostimulation therapy session.

FIG. 8 is a flowchart that describes a method for providing a desired current output of an electrostimulation waveform.

FIG. 9 is a flowchart that describes a technique for responding to rapid changes in load impedance at an electrode-skin interface.

FIG. 10 illustrates generally an example of a block diagram of a machine.

### DETAILED DESCRIPTION

Skin surface electrodes can be used to perform an electrostimulation treatment or diagnostic procedure such as a part of a medical system for delivering electrical electrostimulation or recording electrical response activity. For example, electrodes can be used with an electrostimulation electronics unit for delivering transcutaneous electrostimulation. Here, electrical impulses can be delivered from the electrodes such as to mimic or elicit a neural action potential. For example, for pain therapy, electrical impulses can be delivered below a motor-threshold such as in a manner such that muscles do not contract as a result of the electrical impulses. In other approaches, such as physical rehabilitation, physical therapy, or muscle recovery techniques, electrical impulses can be delivered above a motor-threshold, such as in for causing the muscles to contract.

A challenge in providing an electrostimulation waveform that is effective for treating RLS and PLMD, but that also is comfortable and not disruptive to the subject, is to avoid large changes in current intensity as a result of changes in impedance at the electrode-skin junction. For example, as indicated above, RLS and PLMD can occur during deep sleep and can cause insomnia. In certain instances when a subject moves, the impedance at the electrode-skin interface can change. For example, when the subject rolls over in bed, the impedance at the electrode-skin interface can increase by as much as five or ten times, which can cause a change in the current intensity of an electrostimulation waveform that can be transmitted through the electrodes and that is received by the same electrodes. The change in current intensity in turn can cause discomfort or disruptive effects on the subject. For such electrodes, if impedance increases sufficiently, the level of current delivered through the electrode may also decrease. This can also be problematic because the current delivered through the electrodes can be insufficient to produce sufficient effects, such as to cause muscle contraction, e.g., or to provide effective therapies.

This document describes an external nerve stimulator for treating Restless Legs Syndrome, e.g., at a lower extremity of a patient. An external nerve stimulator for Restless Legs Syndrome can include, e.g., a prescription device including at least one external electrical stimulator or cutaneous electrode to stimulate nerves in a lower extremity of a human patient (e.g., peroneal nerves) and evoke tonic, sustained muscle activation in the legs of the patient such as to help reduce certain symptoms of Restless Legs Syndrome. The present inventors have determined an approach for counteracting rapid changes in impedance experienced by an electrostimulation device. In particular, an electrostimulation device can be configured to provide or “sustain” a desired current output of an electrostimulation waveform despite rapid changes in impedance as experienced by the device. For example, a device can be worn by a subject and can be configured to sense rapid changes in impedance at an electrode-skin junction. The device can then be configured to respond to the rapid changes in impedance and to adjust one or more parameters of an applied electrostimulation waveform. Such adjusting of the waveform can help limit changes in current delivered to the skin of the subject, which can help the patient stay asleep and avoid uncomfortable sensations. For example, the electrostimulation device can generate a controlled current electrostimulation waveform and a controlled voltage electrostimulation waveform, each of which can be delivered to a patient in a charge-balanced manner. The electrostimulation device can include first and second amplifiers electrically connected in a bridge-tied-load (BTL) configuration. The first amplifier can receive differential current signal for the control waveform and can incorporate feedback mechanisms to adapt to impedance changes at the electrode terminals. The electrostimulation device can also include a soft-start ramp generator for bias voltage adjustments and protection diodes to limit overvoltage spikes at or near the electrode terminals.

The present inventors have also determined an approach for regulating the parameters of an electrostimulation waveform such as to promote tonic motor activation at a target nerve (e.g., the peroneal nerve) of a patient's leg. In particular, an electrostimulation device can be configured to provide or “sustain” a desired current output of an electrostimulation waveform and regulate or control any charge dispersed to the patient's skin below a specified value. Providing accurate and relatively constant parameters of the electrostimulation waveform delivered to the skin of the subject can help maintain a desired level of electrostimulation such as to achieve desired therapeutic benefits without being disruptive to the subject's sleep.

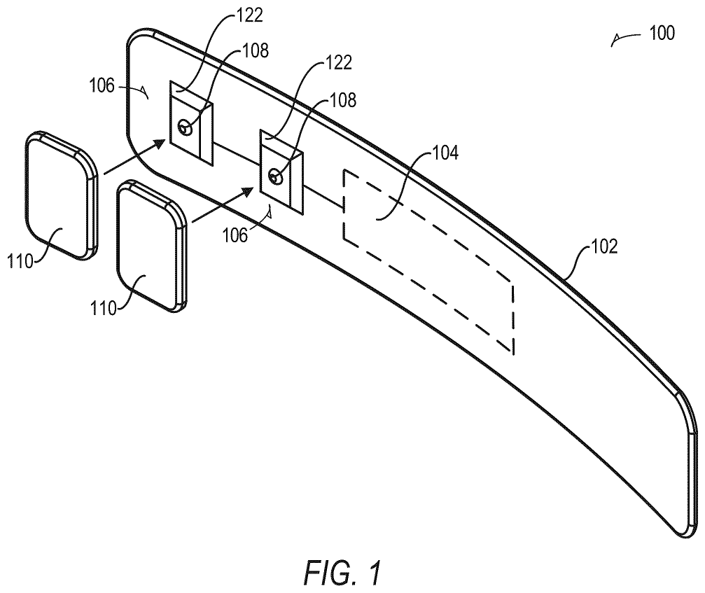

FIG. 1 depicts a perspective view of a wearable electrostimulation device. In an example, an electrostimulation therapy system can include a wearable electrostimulation device 102, an electrostimulation electronics unit 104, and one or more electrostimulation electrodes 106. The wearable electrostimulation therapy device 102 can function to deliver electrostimulation therapy to skin of a subject via the electrode pads 110 which help form the electrodes 106. In an example, the electrode pads 110 can be removably couplable to the wearable electrostimulation device 102. The electrode pads 110 can each be attached to a pairing surface 122 of the wearable electrostimulation device 102. The pairing surface 122 can include the electrode terminal 108 e.g., disposed therein, and the electrode terminal 108 can be electrically connected to the electrostimulation electronics unit 104. In an example, two or more electrode pads 110 are each paired to corresponding pairing surfaces 122 including one electrode terminals 108. In another example, one electrode pad 110 can be paired to an electrode pairing surface 122 containing more than one electrode terminal 108, or one electrode pad can span multiple electrode pairing surfaces 122 containing one or more electrode terminals 108. The electrode pad 110 can be removed for, e.g., hygienic maintenance, electrode maintenance such as rehydrating, or disposal.

The wearable electrostimulation device 102 can be worn by the subject and can include or use the electrostimulation electronics unit 104 coupled to the electrodes 106, such as for transcutaneously delivering an electrostimulation signal. Herein, the term electrostimulation can generally refer to types of therapy that can include transcutaneous electrical nerve stimulation (TENS), electrical muscle stimulation (EMS), electrical stimulation (e-stim), or electrical electrostimulation. The wearable electrostimulation device 102 can be sized and shaped to be able to be attached or held to a body location of the subject, e.g., a leg, arm, foot, waist, neck, head, or chest of the subject. In an example, the wearable electrostimulation device 102 can include or use a strap to help hold the electrodes 106 to the skin of the subject. While electrodes are generally described herein with a focus on providing electrostimulation to a subject, the electrodes can alternatively or additionally be used such as to help detect or measure one or more biosignals or biopotentials from the subject. A particular electrode 106 can include or use an electrode terminal 108 and an electrode pad 110. For example, the electrode terminal 108 can receive a capacitively-coupled (e.g., coupled using series DC-blocking capacitors, e.g., in a charge-balanced arrangement) electrostimulation signal from the electrostimulation electronics unit 104, and can deliver a resulting electrostimulation signal to the skin of the subject, such as via the electrode pad 110. In an example, multiple electrodes, such as two electrodes 106, can be used. For a bipolar electrode example having two electrodes 106, this can a first electrode that can serve as an anode and a second electrode, such as which can serve as a cathode. Also, a plurality of electrodes 106 can be arranged to form a multi-electrode group, matrix, or array such as for one or both of sensing or for delivering the electrostimulation signal to the skin of the subject. In an example, each electrode terminal 108 can be an electrode contact fixed to the wearable electrostimulation device 102 and each corresponding electrode pad 110 can be removably couplable to the device 102. In other examples, the electrode pad can be fixed to the wearable electrostimulation device 102. Because the current density of the electrostimulation signal at the electrode terminal 108 may be larger than desired, the electrode pad 110 can include an embedded or other arrangement of electrical conductors that can help distribute the electrostimulation signal current over a larger effective surface area for delivery to the subject at the skin-electrode interface. The electrode pad can be formed of a hydrogel, a hydrophilic polymer such as polyvinyl alcohol (PVA), carbon, textiles, or other types of conductive or dielectric gels, polymers, or textiles.

The electrode terminal 108 can supply an alternating current (AC) electrostimulation signal for delivery to the skin by the electrode 106. The electrostimulation signal can be supplied by the electrode terminal 108 at a frequency between about 4 kHz to about 15 kHz such as for treating Restless Leg Syndrome (RLS) or Periodic Limb Movement Disorder (PLMD), such as described in Charlesworth U.S. Pat. No. 11,103,691, which is hereby incorporated by reference herein. The wearable electrostimulation device 102 can include or use capacitive coupling such as to inhibit a direct current (DC) signal at the electrode terminal 108 while communicating an AC signal. In an example, the capacitive coupling includes one or more capacitors included in series between each electrode terminal 108 and the electrostimulation electronics unit 104. The one or more capacitors can also be included in series between each electrode terminal 108 and the battery of the electrostimulation electronics unit 104. The electrostimulation signal can be delivered from the electrode terminal 108 to the skin through the electrode pad 110 disposed therebetween.

In an example, the wearable electrostimulation device can include specified control parameters to help promote the safety and effectiveness of the device for neural electrostimulation. Exemplary challenges and mitigation measures associated with other electrostimulation devices are summarized in Table 1, below.

TABLE 1

| Identified Challenges | Mitigation Measures |
|---|---|
| Possible adverse tissue reaction | Biocompatibility evaluation |
| Possible skin discomfort, burns, | Electromagnetic compatibility testing Electrical, |
| electrical shock, or pain at | mechanical, and thermal safety testing non-clinical |
| stimulation site | performance testing Software verification, validation, |
| | and hazard analysis Labeling |
| Possible worsening of Restless Legs | Non-clinical performance testing Software verification, |
| Syndrome (RLS) symptoms, | validation, and hazard analysis Labeling |
| disrupted sleep | |
| Possible user error or device failure | Electromagnetic compatibility (EMC) testing Software |
| due to interference with other | verification, validation, and hazard analysis Labeling |
| devices, leading to delayed or | |
| ineffective treatment | |

In an example, wearable electrostimulation device can be configured subject to any one of the following parameters, separately or in combination with one another:
(1) Non-clinical performance testing can demonstrate that the device performs as intended under anticipated conditions of use. Such testing can include any one or multiple of items i-iii below:
(i) Characterization of the electrical stimulation parameters, including the following: waveforms; output modes; maximum output voltage and maximum output current (e.g., at about 500Ω, about 2 kΩ, and about 10 kΩ loads); pulse duration; frequency; net charge per pulse; maximum phase charge, maximum current density, maximum average current, and maximum average power density (e.g., at about 500Ω), (ii) Characterization of the therapy output across sudden and rapid changes in load impedance, or (iii) Characterization of electrode performance, including the electrical performance, adhesive integrity, shelf-life, reusability, and variation of impedance over the use of therapy.
(2) The tissue-contacting components of the device can be demonstrated to be biocompatible.
(3) Performance testing demonstrates electrical, thermal, and mechanical safety along with electromagnetic compatibility (EMC) of the device in the intended use environment.
(4) Software verification, validation, and hazard analysis can be performed.
(5) Physician and patient labeling can include any one or multiple of items a-f below: (a) Recommended treatment regimes, including frequency and duration of use, and identification of application site(s), (b) Typical sensations experienced during treatment, (c) Methods for identifying the appropriate stimulation intensity that is needed to reduce symptoms of Restless Legs Syndrome and is tolerable to patients, (d) A shelf life for the electrode and reuse information, (e) Summaries of the electrical stimulation parameters and device technical parameters (including any wireless specifications), and (f) Instructions on how to maintain the device, including all user-interface components.

In an example, the wearable electrostimulation device can be a prescription device, e.g., complying with 21 CFR 801.109.

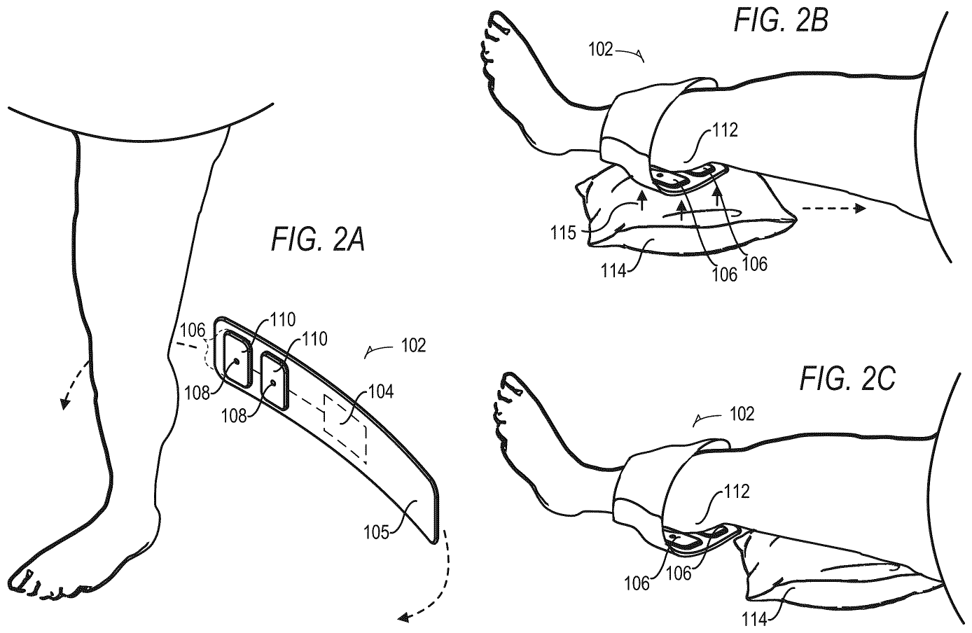

FIG. 2A, FIG. 2B, and FIG. 2C depict an example of a wearable electrostimulation device in use on a subject. An electrostimulation therapy system can include a wearable electrostimulation device 102, an electrostimulation electronics unit 104, and one or more electrodes 106. The electrostimulation therapy device 102 can deliver electrostimulation therapy to the skin of a subject via the electrodes 106, and the device can be configured for delivering such therapy over multiple therapy sessions. As shown in FIG. 2A, the wearable electrostimulation device 102 can include or use a strap, sleeve, clamp, or band 105 to help hold the electrodes 106 to the skin of the subject. In another example, the electrostimulation device can include or use an adhesive or can connect to other items wearable by the subject, e.g., hats, clothing, etc. Alternatively, the wearable electrostimulation device 102 can be sufficiently wearable on the skin surface of the subject by adhesion forces of the electrodes 106 alone without the need for additional features to help hold the device 102 to the subject. The wearable electrostimulation device 102 can be attached or held to a body location of the subject, e.g., a leg, arm, foot, waist, neck, head, or chest of the subject at or near a nerve location of the subject skin for transcutaneous electrostimulation thereof.

As depicted in FIG. 2B, an individual electrostimulation therapy session can be performed with the subject lying down, e.g., during a sleep session. The one or more electrostimulation electrodes 106 can be applied at an external target body location 112 of a subject. During the electrostimulation therapy session, the one or more electrodes 106 can receive or sense a first indication of a first impedance between the respective electrode 106 and the external target body location 112. The electrostimulation electronics unit 104 can generate a high-frequency (HF) electrostimulation waveform for delivery, via the one or more electrodes 106, to the external target body location 112. Here, the electrostimulation waveform can be generated by the electrostimulation electronics unit 104 in accordance with the sensed indication of the first impedance. The waveform can be generated at a frequency between 300 Hertz (Hz) and 10,000 Hz and at a specified first current between 5 milliAmps (mA) and 50 mA. The waveform delivered to the target body location 112 of the subject by the electrodes 106 can be alternating current (AC) waveform, and the AC waveform can be significantly charge-balanced. In an example, the electrostimulation electronics unit 104 can include capacitive-coupling circuitry such as capacitors in series for distributing AC signal to the skin surface of the subject without distributing significant amounts of direct current (DC) signal to the skin surface of the subject.

As depicted in FIG. 2A and FIG. 2B, an external object 114, e.g., a pillow, bedding, clothing, or bedmate, can shift with respect to the subject during a sleep session. This can involve a change in forces 115 applied to the device 102 and in turn can alter a force that the one or more electrodes 106 contact the target body location 112. Such changes in force can be detected by the electrostimulation electronics unit 104, such as detected by sensing changes in impedance or detection via an accelerometer, and the electrostimulation waveform can be adjusted accordingly. For example, during the electrostimulation therapy session, the one or more electrodes 106 can receive or sense a second indication of a second impedance between the respective electrode 106 and the external target body location 112. Here, the second impedance can be significantly different from the first impedance, such as greater than a specified threshold difference. The electrostimulation electronics unit 104 can include waveform modulation circuitry to adjust at least one parameter of the at least one HF electrostimulation waveform to adjust a current of the electrostimulation waveform toward the specified first current between 5 mA and 50 mA. This can help avoid significant deviations in a current of the waveform corresponding with a change in impedance, e.g., caused by the moving of the external object 114 with respect to the subject.

As discussed further below, one or both of the respective first and second indications of the first and second impedances can be sensed using impedance detection circuitry. Also, the one or both of respective first and second indications of the first and second impedances can be received, at least in part, by monitoring the output current of the waveform from the electrostimulation electronics unit 104. In addition, one or both of the respective first and second indications of the first and second impedances can be received, at least in part, from monitoring the phase angle between the output current and the electrostimulation waveform. One of both of the first and second indications of the first and second impedances can then be used, as further described below, to calculate a change in impedance between the first and second impedances.

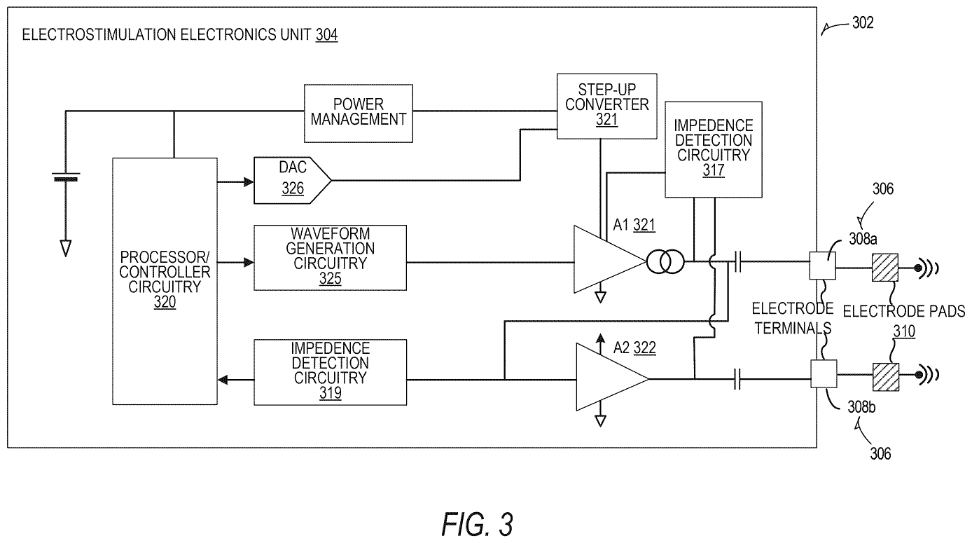

FIG. 3 is a schematic representation of portions of an example of a wearable electrostimulation device. The wearable electrostimulation device 302 can include an electrostimulation electronics unit 304, electrode terminals 308, and electrode pads 310. The wearable electrostimulation device 302 can be similar in many respects to wearable electrostimulation device 102. The components, structures, configuration, functions, etc. of system 300 can therefore be the same as or substantially similar to that described in detail above with reference to systems 100 and 300. The electrostimulation electronics unit 304 can be configured to produce a controlled-current waveform (e.g., specified constant current amplitude AC electrostimulations) across a varying impedance present at an interface between the electrostimulation electrodes 306 and the skin or other tissue of the subject contacted by such electrostimulation electrodes 306.

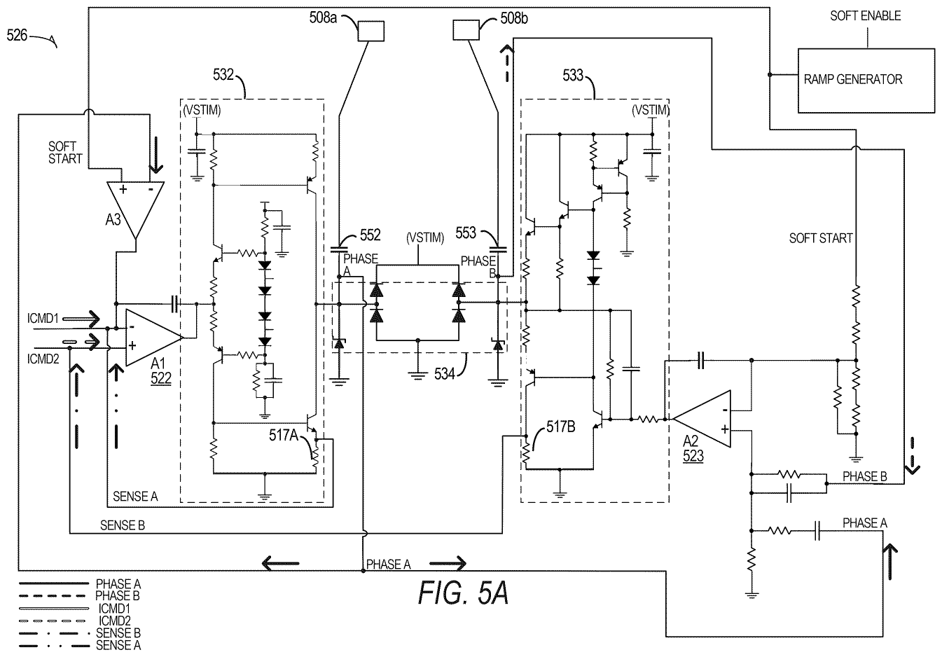

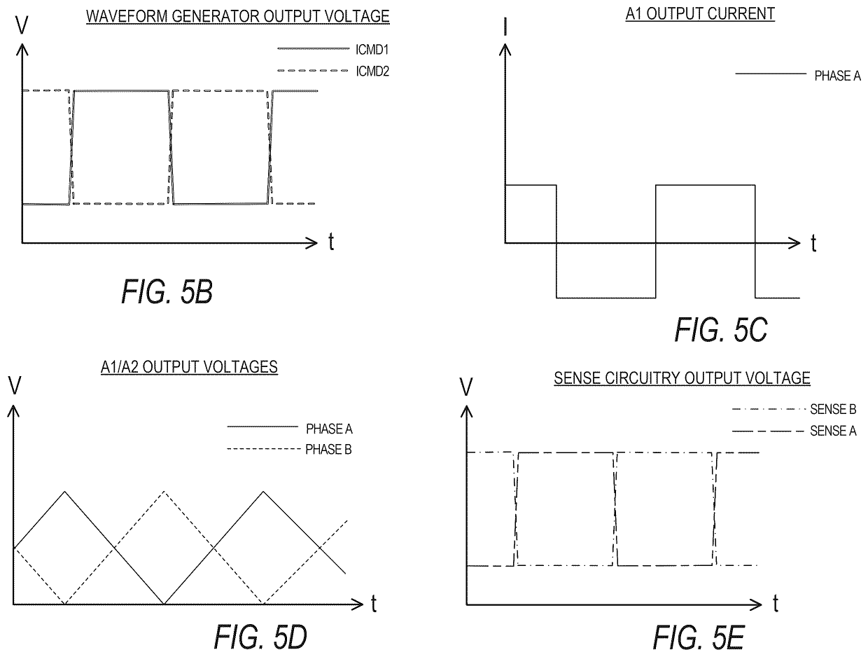

The electrostimulation electronics unit 304 can include a first amplifier A1 322 and a second amplifier A2 323 arranged to supply an electrostimulation waveform to first and second electrode terminals 308a and 308b, respectively. For example, the first amplifier A1 322 can include a transconductance amplifier that can produce a first output waveform with a controlled or constant current in response to its input voltage signal. The second amplifier A2 can include a voltage amplifier that can produce a different second output waveform with a controlled or constant voltage in response to its input voltage signal. By driving the electrostimulation electrodes 306 via separate amplifiers, A1 322 and A2 323, the electrostimulation signal waveforms at the electrode terminals 308a and 308b can be smoothly controlled to be responsive to sudden changes in load impedance—without introducing ringing or other oscillations, e.g., such as may be caused by other approaches including a single amplifier or two constant-current amplifiers counteracting each other through a capacitive load. For example, the first amplifier A1 322 can be a transconductance amplifier and can be arranged to receive, as its input voltage, an output voltage from waveform generation circuitry 325 (similar to the control waveform depicted in FIG. 5B). The first amplifier A1 322 can establish a desired electrostimulation current level based on feedback providing information about a load impedance, such as can be detected by a first impedance detection circuit 317. In an example, the first impedance detection circuit 317 can include one or more current sensing resistors, such as can be used to sense a current therethrough at an output of the first amplifier A1 322, the second amplifier A2 323, or both of these. An indication of the current through the current sensing resistors can be determined by measuring a voltage signal across a respective current sensing resistor. The current-indicating feedback voltage signal can be summed as a feedback signal with the output voltage from the waveform generation circuitry 325 being provided as an input to the amplifier A1 322, such as to maintain a relatively constant or stable current output from the amplifier A1 322 even during rapid load impedance changes such as may occur at an interface between an electrode pad and skin of the patient. In an example, the waveform generation circuitry 325 can establish the output voltage at a reference voltage (similar to waveform depicted in FIG. 5B) based on a second impedance detection circuit 319. The output current of the first amplifier A1 (similar waveform depicted below in FIG. 5C) can be supplied to the first electrode terminal 308a and the voltage at the output node of the first amplifier A1 can be used as an input into the second amplifier A2 323. In using the output voltage of the first amplifier A1 322 as an input to the second amplifier A2 323, the first and second amplifiers 322 and 323 can deliver relatively complementary output voltage waveforms, inverted in sign with respect to each other (similar to waveforms depicted in FIG. 5D). Such inverted waveforms can refer to waveforms separated by a phase shift of about 180 degrees. The electrostimulation electronics unit 304 can also include a first impedance detection circuit 317, e.g., configured for sensing or receiving an indication of a change in load impedance or an electrode-skin interface impedance at or near one or more of the electrostimulation electrodes 306. To help produce a constant-current output at the electrode terminals 308a and 308b, the first impedance detection circuit 317 can provide a negative feedback loop from the output current of the first amplifier A1 322, the second amplifier A2 323, or both back to the first amplifier A1 322. In an example, the output current can be sensed by one or more sense resistors that can be included in the first impedance detection circuit 317. In the event of detecting an overcurrent spike exceeding a threshold current at the output of the first amplifier A1 322 or the second amplifier A2 323, the first amplifier A1 322 can attenuate the output current below the threshold current.

The electrostimulation electronics unit 304 can also include or use waveform generation circuitry 325 that can be included in or coupled to the controller circuitry 320. In an example, the waveform generation circuitry 325 can include waveform modulation circuitry or at least one waveform modulation component. Here, the waveform generation circuitry 325 can adjust, in response to a detected or sensed indication of a threshold change in impedance, at least one parameter of the at least one HF electrostimulation waveform to adjust a current of the electrostimulation waveform. For example, the waveform generation circuitry 325 can facilitate changing a voltage parameter of the waveform to maintain a specified current amplitude despite a significant change in impedance. Alternatively or additionally, a sensed impedance detection voltage from the first impedance detection circuit can be summed with a control signal from the waveform generation circuitry 325 to modulate the control waveform, according to sensed impedance, in an analog fashion. In an example, the battery can be electrically connected or otherwise interfaced to a step-up power converter circuit, such as can generate a programmable output DC voltage (e.g., 12V, 20V, 30V, or other specified DC output voltage. The DC output voltage needed can be determined by the controller circuit 320, such as based on a sensed load impedance detected using the first impedance detection circuit 317, or a second impedance detection circuit 319, described below.

In an example, the controller circuitry 320 can use a sensed indication of load impedance (from at least one of first or second impedance detection circuit 317 or 319, which can be used by the controller circuitry 320 in combination with other information (e.g., accelerometer, sEMG, or temperature data) such as to determine the magnitude of the DC output voltage of the step-up converter 321 needed to generate the desired electrostimulations. Determining the magnitude of the DC output voltage of the step-up converter 321 can help determine parameters to conserve battery power while providing or increasing therapeutic efficacy of the electrostimulations. The controller circuitry 320 can establish one or more patterns of the desired electrostimulation, such as by using one or more stored electrostimulation waveform parameters that can be generated by the controller circuitry 320. The controller circuitry 320 can use the one or more stored electrostimulation parameters, such as to generate one or more analog electrostimulation control voltage waveforms, such as using a digital-to-analog (D/A) converter. The resulting one or more generated analog electrostimulation control voltage waveforms can be converted to a proportionate, controlled, load-independent current. The resulting controlled-current electrostimulation signals can be routed, e.g., via an electrode interface 324, to a desired corresponding electrode terminal 308 and distributed to the skin of a subject via the corresponding electrode pad 310.

The electrostimulation electronics unit 304 can also include a second impedance detection circuit 319, e.g., configured for sensing or receiving an indication of a change in load impedance or an electrode-skin interface impedance at or near one or more of the electrostimulation electrodes 306. In an example, such impedance information, e.g., including respective first and second indications of different first and second impedances, can be provided to processor/controller circuitry 320 for electrostimulation waveform parameter selection or adjustment. For example, the second impedance detection circuit 319 can measure a voltage of the constant current output waveform of the first amplifier A1 322 and detect a peak in voltage. Effectively, the impedance detection circuitry can use the detected peak in voltage to sense the indication of a change in load impedance or an electrode-skin interface impedance at or near one or more of the electrostimulation electrodes 306. In the event of a detecting an overvoltage peak at the second impedance detection circuit 319, the voltage supplied to the step-up converter 321, such as via a digital to analog converter (DAC) 326, can be adjusted to dynamically change the supply voltage to the first amplifier A1 322. If, in the event of the detection of the overvoltage peak, the step-up converter 321 and the DAC 326 have achieved a threshold output voltage, the processor/controller circuitry can determine that the load impedance has surpassed an expected threshold value and a voltage, current, or both supplied by the waveform generation circuitry 325 can be reduced to counteract the change in impedance. This can help control a perceived electrostimulation sensation, felt by the subject, at the external target body location during an event of a sudden or rapid change in load impedance. In an example, the second impedance detection circuit 319 can include one or more current sensing resistors, such as can sense a current at the electrodes. The current sensed by the current sensing resistors can be converted by the current sensing resistors into a voltage signal. The resulting voltage signal can be received at one or more inputs of a detection signal amplifier and can be buffered or amplified by the detection signal amplifier. The resulting buffered or amplified voltage signal can be digitized, such as by an analog-to-digital converter (ADC) circuit, such as can be included in or coupled to the controller circuitry 320. In another example, the impedance detection circuitry 319 can include a capacitive element, such as can be configured to sense or receive an indication of a change in load impedance or an electrode-skin interface impedance at or near one or more of the electrostimulation electrodes 306 by measuring a change in capacitance at the interface between the electrodes 306 and the skin or other tissue of the subject. Regardless of the inclusion of the second impedance detection circuit 319 and as detailed further below with respect to FIG. 5, the electrostimulation electronics unit 304 can adjust an output voltage or current in response to a sudden or rapid change in load impedance, independent of or in absence of feedback from the second impedance detection circuit 319, such as exclusively in an analog fashion via the first impedance detection circuit 317.

In an example, the electrostimulation electronics unit can generate and deliver an electrostimulation waveform at a specified first current between 5 mA and 50 mA. For example, the specified first current can be within a range of about 25 mA and about 35 mA. The specified first current can be determined based on an efficacy of a particular current at treating at least one symptom of RLS or PLMD or determined tolerance of the subject. The specified first current can also be a benchmark or target current, and the benchmark current can be adjusted during an electrostimulation therapy session independent from the waveform generation circuitry 325 which facilitates constant-current output. In other words, the waveform generation circuitry 325 can adjust a current of the electrostimulation waveform toward a benchmark current, in response to a sensed or inferred impedance change, while the benchmark current can be shifted (e.g., as a moving target for the waveform generation circuitry 325) based on other therapy determinations such as efficacy and tolerance with respect to a particular subject.

In an example, the waveform generation circuitry 325 can facilitate adjustment of the electrostimulation waveform, such that an output current is reverted or restored to the specified first current, within about 35 microseconds (μs) of a threshold change in load impedance at electrostimulation electrodes of the wearable electrostimulation device. In another example, the waveform generation circuitry 325 can facilitate adjustment of the electrostimulation waveform, such that an output current is reverted or restored to the specified first current, within about 5 μs of a threshold change in load impedance at electrostimulation electrodes of the wearable electrostimulation device.

In the time period where the waveform generation circuitry 325 rapidly adjusts the electrostimulation waveform (e.g., within about 35 μs), the electrostimulation electronics unit 304 can also include voltage clamp componentry configured to limit overcurrent peaking of the waveform before the waveform returns to desired parameters. Overcurrent peaking can occur where the electrostimulation waveform rapidly increases to a peak, such as a relatively high positive or negative peak. Current peaking refers to the occurrence of a peak current that exceeds the specified first current, which can be uncomfortable for the subject. For example, the voltage clamp componentry can control a maximum overcurrent peak to remain less than 600% of the specified first current before adjusting the current toward the specified first current. The voltage clamp componentry can control a maximum overcurrent peak to remain less than 300% of the specified first current before adjusting the current toward the specified first current. The voltage clamp componentry can also control a maximum overcurrent peak such that the peak transfers less than 800 nanocoulombs (nC) of charge to the external target body location before the current of the electrostimulation waveform can be reverted to the specified first current.

In an example, the electrostimulation electronics unit 304 can trigger adjusting at least one parameter of the electrostimulation waveform, in response to sensed or inferred impedance change where the impedance change reaches at least a specified threshold value or a specified threshold condition. The specified threshold condition can refer to an impedance change of less than ±20 ohms (Ω), e.g., between about ±5Ω and about ±15Ω. The specified threshold condition can be where a first indication of a first impedance is at a value indicating an open circuit and a second indication of a second impedance is within a range of about 300Ω and about 600Ω. The specified threshold condition can be where a first indication of a first impedance is within a range of about 300Ω and about 600Ω and a second indication of a second impedance is at a value indicating an open circuit. The specified threshold condition can be where a first indication of a first impedance is within a range of about 300Ω and about 600Ω and a second indication of a second impedance is at a value indicating a short circuit. The specified threshold condition can be where a first indication of a first impedance is at a value indicating a short circuit and a second indication of a second impedance is within a range of about 300Ω and about 600Ω. The specified threshold condition can be where a first indication of a first impedance is within a range of about 900Ω and about 1.1 kiloohms (kΩ) and a second indication of a second impedance is within a range of about 90Ω and about 610Ω. The specified threshold condition can be where a first indication of a first impedance is within a range of about 90Ω and about 610Ω and a second indication of a second impedance is within a range of about 900Ω and about 1.1 kiloohms (kΩ). In an example, the electrostimulation electronics unit 304 can trigger adjusting at least one parameter of the electrostimulation waveform, in response to sensed or inferred impedance change where the impedance change fulfills at least one of a plurality of different specified threshold conditions, such as a combination of the exemplary specified threshold values above.

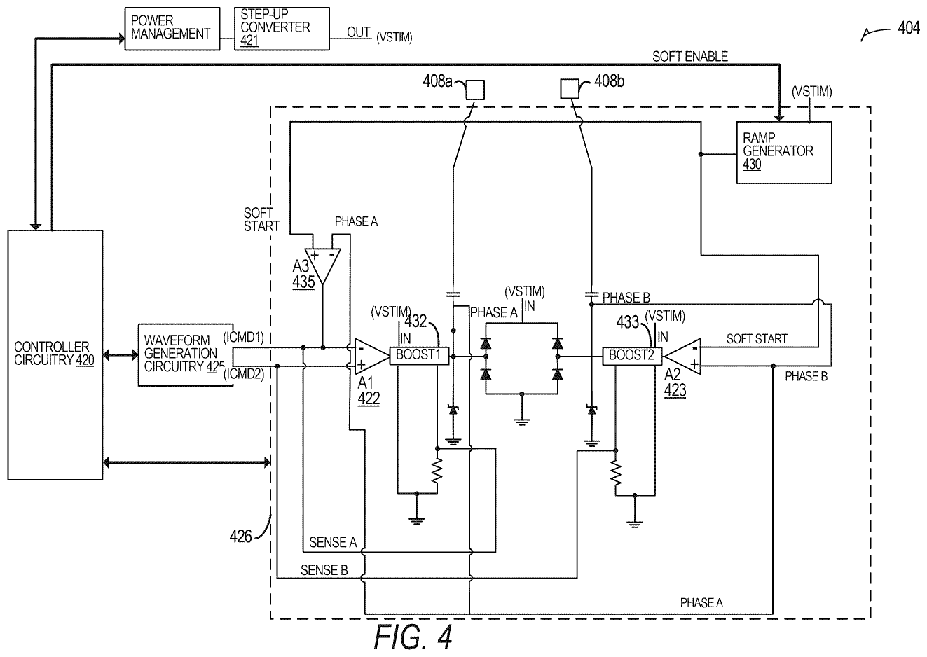

FIG. 4 is a schematic representation of portions of an example of an electrostimulation electronics unit of a wearable electrostimulation device. The electrostimulation electronics unit 404 can be substantially similar to the electrostimulation electronics unit 304 of FIG. 3. The components, structures, configurations, functions, etc. of electrostimulation electronics unit 404 can therefore be the same as or substantially similar to that described in detail with respect to electrostimulation electronics unit 304. In an example, the electrostimulation electronics unit 404 can include controller circuitry 420, a step-up converter 421, waveform generation circuitry 425, and an electrostimulation signal amplification unit 426. The amplification unit 426 can include first and second amplifiers A1 422 and A2 423, a ramp generator, and first and second boost circuits 432 and 433, each corresponding with or included in A1 422 and A2 423, respectively. The arrangement of the amplification unit 426 facilitates rapid (e.g., within less than about 30 microseconds (μs) or less than about 5 μs) response to changes in impedance at an electrode-skin junction (e.g., caused by patient movement or disconnecting of an electrode from patient tissue). Such an arrangement can facilitate feedback, sensed at the first and second boost circuits 432 and 433 as an indication of electrode-skin impedance, to modulate operation of the first amplifier A1 422 in an analog fashion. Such adjusting of the waveform can help limit changes in current (e.g., maximum overcurrent peak remaining less than about 600% or less than about 300% of a specified previous current) delivered to the skin of the subject, which can help the patient stay asleep and avoid uncomfortable sensations.

In an example, A1 422 can receive one or more control waveforms (e.g., current command waveform 1 (“ICMD1”), current command waveform 2 (“ICMD2”), or both) from the waveform generation circuitry. For example, A1 422 can receive ICMD1 and ICMD2 respectively at inverting and non-inverting input terminals of A1 422. The one or more control waveforms can each be AC waveforms and can control operation of A1 422 by modulation of a current of the one or more control waveforms. Thus, an output signal current of A1 422 can be substantially current-controlled, e.g., via the waveform generation circuitry 425. As described further below with respect to FIG. 5, the one or more control waveforms such as ICMD1 and ICMD2 can respectively be summed with or otherwise modulated by feedback signals (e.g., Sense A and Sense B) from corresponding first and second boost circuits 432 and 433.

Subject to the one or more control waveforms, A1 422 can supply a controlled current first electrostimulation output waveform via a first electrode terminal 408a. In an example, A1 422 and A2 423 can be electrically connected in a bridge-tied load (BTL) configuration. An output of A1 422 (“phase A”) can be supplied to an input terminal (e.g., a non-inverting input terminal) of A2 423. As such, A2 423 can be substantially voltage-controlled based on the constant-current output of A1 423. A2 423 can supply a controlled voltage second electrostimulation output waveform via a second electrode terminal 408b. In an example, a signal at an output of A2 423 (“phase B”) can be fed back to an input terminal (e.g., a non-inverting input terminal) of A2 423. Such a feedback loop within A2 423 can help facilitate frequency-matching between the first and second electrostimulation output waveforms.

Each of the ramp generator 430, the first boost circuit 432, and the second boost circuit 433 can receive a direct current (DC) power waveform (“VSTIM”) from the step-up converter 421. In an example, the output voltage of the step-up converter 421 can be adjusted based on an electrical load (e.g., a load at the first or second boost circuits 432 or 433, a load at the ramp generator, or both). The first and second boost circuits can level-shift and translate respective outputs from the first and second amplifiers 422 and 423 from amplifier power supply levels to electrostimulation power supply levels. The ramp generator 430 can be electrically connected to the controller circuitry 420, such as to receive a command or other signal for a power-on event (“soft enable”) to ramp up the electrostimulation device. The ramp generator 430 can supply a bias voltage (“soft start”) toward A1 422 (e.g., at an inverting input terminal). In an example, the amplification unit 426 can include a third amplifier A3 435 located between an output of the ramp generator 430 and an inverting input of A1 422. Here, the A3 435 can receive the bias voltage (“soft start”) at a non-inverting first terminal and a boosted output of A1 422 (“phase A”) at a second (inverting) terminal, and can supply a signal to the inverting input of A1 422 based on a difference between the bias voltage (“soft start”) and phase A. The ramp generator 430 can also supply the bias voltage (“soft start”) to an input terminal (e.g., an inverting input terminal) of A2 423 to facilitate a ramping of signal through A2 423 upon receiving the power-on event.

FIG. 5A is a schematic representation of an electrostimulation signal amplification unit of a wearable electrostimulation device. The electrostimulation signal amplification unit 526 can be substantially similar to the electrostimulation signal amplification unit 426 of FIG. 4. The components, structures, configurations, functions, etc. of electrostimulation signal amplification unit 526 can therefore be the same as or substantially similar to that described in detail with respect to electrostimulation signal amplification unit 426. In an example, the amplification unit 526 can include first and second amplifiers A1 522 and A2 523, first and second boost circuits 532 and 533 respectively coupled to A1 522 and A2 523, an arrangement of protection diodes 534, and first and second integration capacitors 552 and 553 respectively coupled to first and second electrode terminals 508a and 508b. FIG. 5A generally shows a relative directionality of currents for each of “Phase A”, “Phase B”, “ICMD1”, “ICMD2”, Sense A”, and “Sense B” nodes of the schematic. Likewise, FIG. 5B, FIG. 5C, FIG. 5D, and FIG. 5E are charts showing waveforms for each of “Phase A”, “Phase B”, “ICMD1”, “ICMD2”, Sense A”, and “Sense B” nodes of the schematic of FIG. 5A.

In an example, the amplification unit 526 can receive at least one alternating current (AC) control waveform, (e.g., ICMD1 and ICMD2), at A1 522. The at least one AC control waveform can include a differential current signal of opposing polarity components that are respectively coupled to first and second inputs of A1 522. As shown in FIG. 5B, the at least one AC control waveform can be a substantially square-wave and include components ICMD1 and ICMD2 with substantially opposing or inverted polarities. In an example, the first and second boost circuits 532 and 533 can comprise different circuit constructions from each other, such that the first boost circuit 532 has a higher output impedance than that of the second boost circuit 533. As shown in FIG. 5D, the output voltages of A1 522 and A2 523 can each form substantially triangular waveforms and can be opposing or inverted polarities with respect to each other. Also, each of the first and second boost circuits 532 and 533 can invert a waveform polarity from that of the output of each respective amplifier, A1 522 and A2 523. In an example, the first and second boost circuits 532 and 533 each include or are coupled to respective first and second current-sensing or shunt resistors 517A and 517B. The current sensing-resistors 517A and 517B can form a similar circuit to that described with respect to the impedance detection circuitry 317 of FIG. 3. As such, “Sense A” and “Sense B” can be supplied to respective input terminals of A1 522 (along with ICMD1 and ICMD2) such that respective feedback indications of current through respective first and second boost circuits 532 and 533, each in accordance with a change of impedance associated with at least one of the first or second patient electrode terminals, to modulate operation of A1 522. As shown in FIG. 5E, the respective feedback indications from the first and second boost circuits 532 and 533 can be substantially square-waveforms and can have substantially opposing or inverted polarities with respect to each other. As such, the amplification unit 526 can, via analog feedback connections and without impedance sensing via a microcontroller, counteract rapid changes in impedance at one or both of the electrode terminals to help ensure a relatively constant-current electrostimulation waveform (e.g., according to the waveform shown in FIG. 5C) is delivered at the electrode-tissue interface.

In an example, the outputs of each of A1 522 and A2 523 can be coupled the first and second integration capacitors 552 and 553, respectively. The first and second integration capacitors 552 and 553 can help limit delivery of any stray DC voltage to the patient tissue via the electrodes. The amplification unit 526 can also include the arrangement of protection diodes 534, located between respective outputs of A1 522 and A2 523. The arrangement of protection diodes can help limit an overvoltage spike at the firs and second patient electrode terminals 508a and 508b with respect to at least one of a DC electrostimulation power supply voltage (e.g., “VSTIM”) or a ground reference voltage.

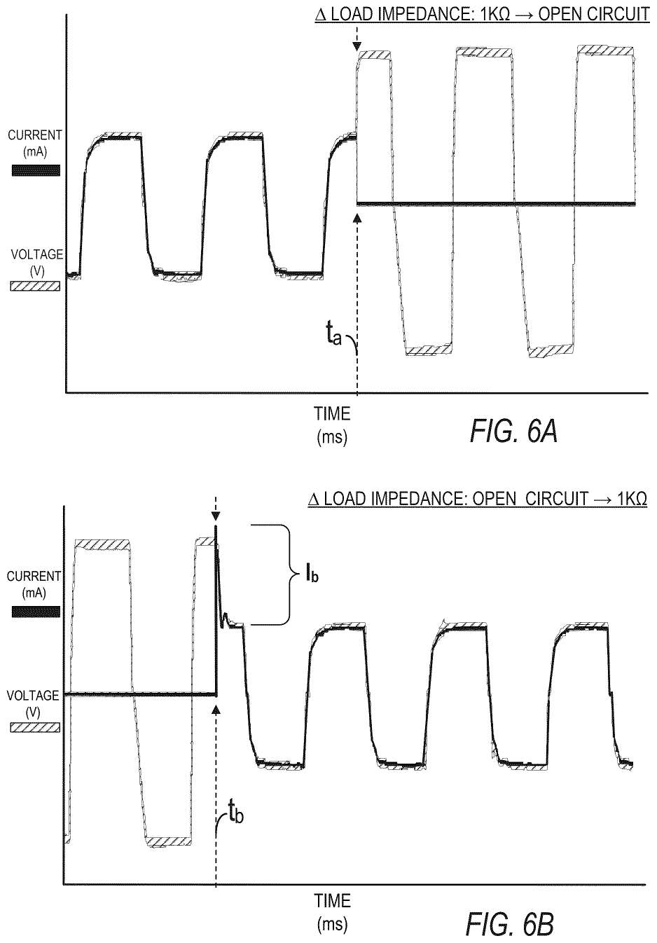

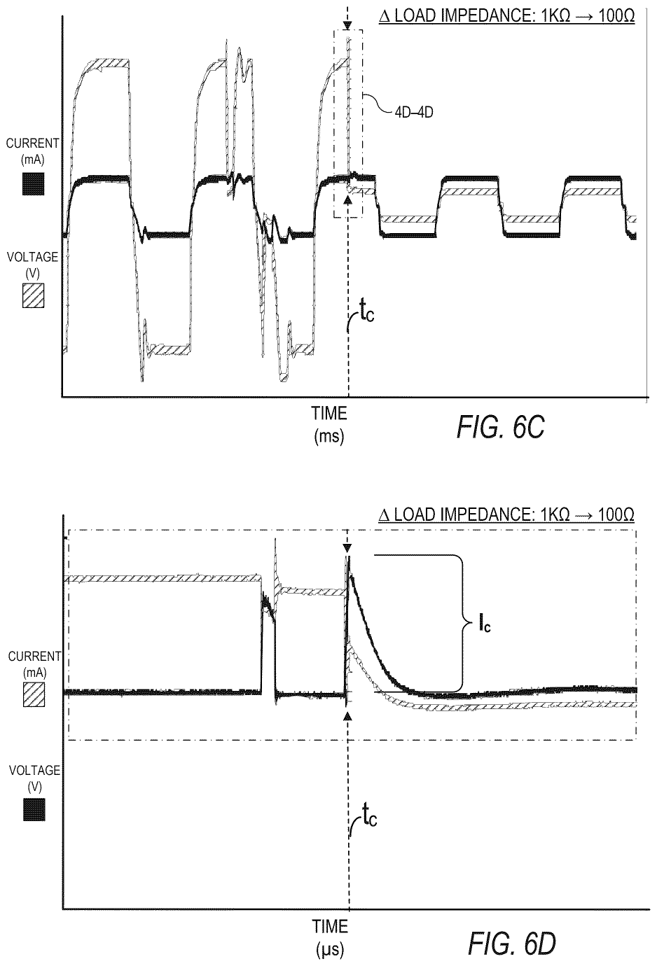

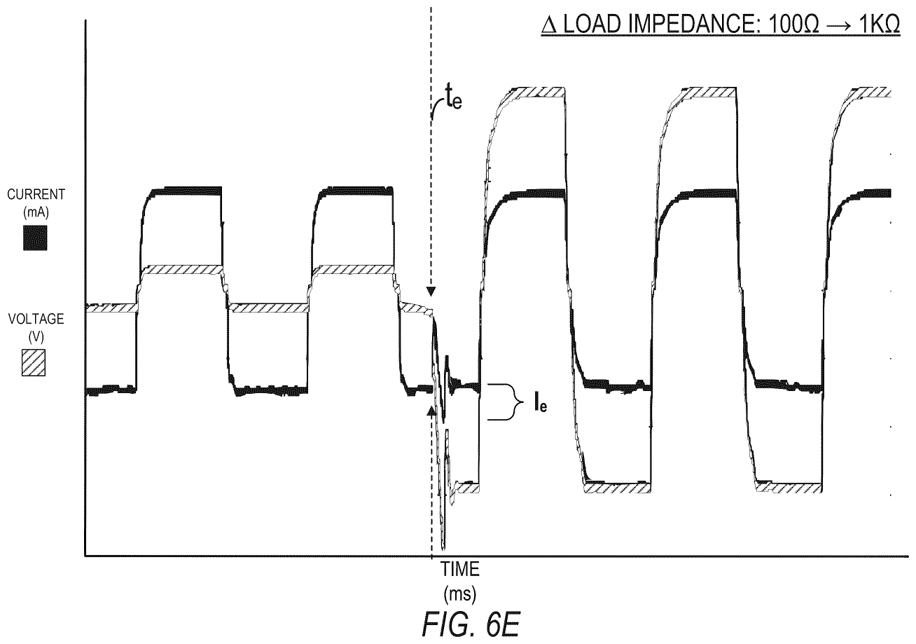

FIG. 6A, FIG. 6B, FIG. 6C, FIG. 6D, and FIG. 6E show examples of oscilloscope waveforms produced by wearable electrostimulation device, each involving a rapid change in impedance.

As depicted in FIG. 6A, a wearable electrostimulation device can encounter a change in load impedance, sensed or inferred, from about 1kΩ toward a value indicating an open circuit at time tₐ. As depicted, the electrostimulation electronics unit 304 (as depicted in FIG. 3) of the wearable electrostimulation device 302 can adjust a voltage waveform amplitude to rapidly (e.g., within about 5 μs) become compliance-voltage limited. In an example, when the electrostimulation electronics unit 304 encounters such a change in load impedance from about 1 kΩ toward a value indicating an open circuit, the electrostimulation electronics unit 304 can adjust at least one parameter of the electrostimulation waveform towards the first specified current without any substantial overcurrent peak.

As depicted in FIG. 6B, a wearable electrostimulation device can encounter a change in load impedance, sensed or inferred, from a value indicating an open circuit toward a value of about 1kΩ at time t_{b}. Here, the electrostimulation electronics unit 304 can limit an overcurrent peak I_{b} to remain less than about 100 mA or less than about 300% of the first specified current. For example, the electrostimulation electronics unit 304 can limit an overcurrent peak I_{b} to about 76 mA or about 250% of the first specified current. The electrostimulation electronics unit 304 can counteract such a change in load impedance, by recovering or reverting the current to the first specified current, in less than about 35 μs, e.g., in less than about 28 μs.

As depicted in FIG. 6C and FIG. 6D, a wearable electrostimulation device can encounter a change in load impedance, sensed or inferred, from a value of about 1 kΩ toward a value of about 100Ω at time tₑ. Here, as shown in the magnified and stretched view of FIG. 6D, the electrostimulation electronics unit 304 can limit an overcurrent peak I_{c} to remain less than about 200 mA or less than about 600% of the first specified current. For example, the electrostimulation electronics unit 304 can limit an overcurrent peak I_{c} to about 170 mA or about 870% of the first specified current. The electrostimulation electronics unit 304 can counteract such a change in load impedance, by recovering or reverting the current to the first specified current, in less than about 5 μs, e.g., in less than about 1 μs.

As depicted in FIG. 6E, a wearable electrostimulation device can encounter a change in load impedance, sensed or inferred, from a value of about 100Ω toward a value of about 1 kΩ at time tₑ. Here, the electrostimulation electronics unit 304 can limit an overcurrent peak Iₑ to remain less than about 50 mA or less than about 200% of the first specified current. For example, the electrostimulation electronics unit 304 can limit an overcurrent peak Iₑ to about 41 mA or about 140% of the first specified current. The electrostimulation electronics unit 304 can counteract such a change in load impedance, by recovering or reverting the current to the first specified current, in less than about 35 μs, e.g., in less than about 32 μs.

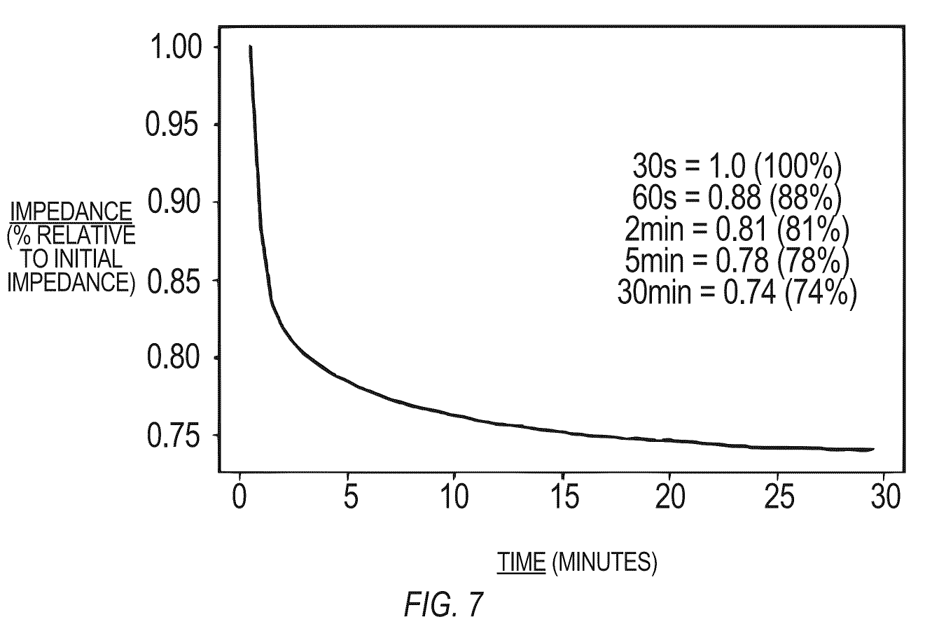

FIG. 7 is a graph depicting an example of an initial impedance drop exhibited by the first and second skin electrodes at a beginning of an electrostimulation therapy session. In an example, the impedance exhibited at the first and second skin electrodes at a target body location can decrease by an expected first percentage between about 20% and about 30% (e.g., about 26%) within the first 30 minutes of an electrostimulation therapy session. For example, between about 80% and about 90% of the first percentage drop can occur within the first 5 minutes of the electrostimulation therapy session. In another example, between about 80% and between about 90% of the first percentage drop can occur within the first 2 minutes of the electrostimulation therapy session, such that the electrostimulation electronics unit 304 (as depicted in FIG. 3) can encounter an impedance varying less than about 10% within the first 2 minutes of the electrostimulation therapy session. Impedances exhibited at the first and second skin electrodes after the first 30 minutes of the electrostimulation therapy session can be relatively consistent, such as varying less than about 5% or varying less than about 10%. In an example, impedances exhibited at the first and second skin electrodes after the first 5 minutes of the electrostimulation therapy session can be relatively consistent, such as varying less than about 5% or varying less than about 10%. In an example, the waveform generation circuitry 325 (as depicted in FIG. 3) can counteract such an expected first percentage impedance drop at the electrostimulation electrodes such as to regulate a controlled-current waveform output within the first 30 minutes of the therapy session. Also, the waveform generation circuitry 325 can counteract such an expected first percentage impedance drop at the electrostimulation electrodes such as to regulate the waveform at an output voltage of between about 18 Volts (V) and about 22 V, an output current between about 35 milliamps (mA) and about 45 mA, and an average power density between about 0.03 Watts per Square Centimeter (W/cm²) and about 0.06 W/cm².

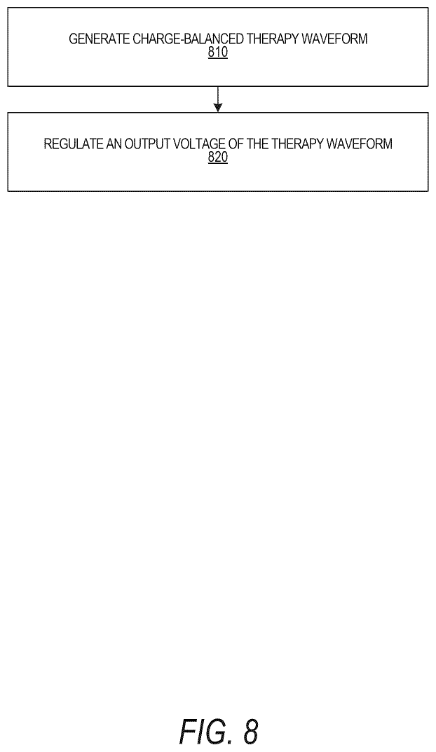

FIG. 8 is a flowchart that describes a method for providing a desired current output of an electrostimulation waveform.

In an example, at 810, the method can include generating, via an electrostimulation electronics unit, a bi-phasic pulsed, charge-balanced therapy waveform at a frequency of between about 800 hertz (Hz) and about 10,000 Hz. For example, generating the bi-phasic pulsed, charge-balanced therapy waveform can include generating waveform at a maximum pulse charge between about 4 microcoulombs (μC) and about 65 μC. In an example, regulating the therapy waveform can include establishing or adjusting a maximum current density between about 2.2 milliAmps per square centimeter (mA/cm²) and about 2.6 mA/cm² when the therapy waveform can be delivered, via the electrostimulation electrodes, at a resistance of at or near 800 ohms (Ω). In an example, the therapy waveform can be a multiphasic waveform including symmetrical anodic and cathodic phases with respective durations between about 120 μs and about 125 μs.

At 820, the method can include regulating, via electrostimulation waveform regulator circuitry, an output voltage of the therapy waveform at between about 18 Volts (V) and about 22 V, an output current at between about 35 milliamps (mA) and about 45 mA, and an average power density at between about 0.03 Watts per Square Centimeter (W/cm²) and about 0.06 W/cm² when the therapy waveform can be delivered, via electrostimulation electrodes, at a resistance of at or near 800 ohms (Ω). For example, regulating the therapy waveform can include establishing or adjusting a maximum output voltage to a value between about 19.5 V and about 20.5 V when the therapy waveform can be delivered, via the electrostimulation electrodes, at a resistance of at or near 800 ohms (Ω) of resistance. In an example, regulating the therapy waveform can include establishing or adjusting a maximum output current to a value between about 38 mA and about 42 mA when the therapy waveform can be delivered, via the electrostimulation electrodes, at a resistance of at or near 800 ohms (Ω). Also, regulating the therapy waveform can include establishing or adjusting a maximum average power density to a value between about 0.045 W/cm² and about 0.05 W/cm² when the therapy waveform can be delivered, via the electrostimulation electrodes, at a resistance of at or near 800 ohms (Ω) of resistance. In an example, generating the bi-phasic pulsed, charge-balanced therapy waveform can include generating waveform pulses at a pulse duration between about 200 microseconds (μs) and about 800 μs.

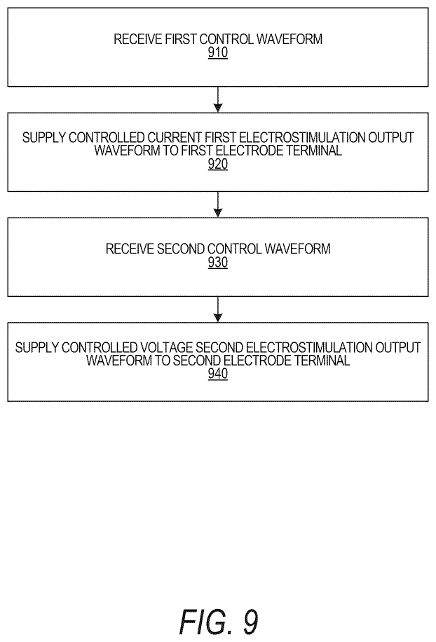

FIG. 9 is a flowchart that describes a technique for responding to rapid changes in load impedance at an electrode-skin interface. The technique can be performed using various electrostimulation devices (e.g., device 100) and electrostimulation electronics units (e.g., unit 304 or 404) described herein.

At 910, a first alternating current (AC) first control waveform, e.g., generated by the control waveform generation circuitry within the electrostimulation device, can be received by a first amplifier. In an example, the AC first control waveform includes a differential current signal with opposing polarity components, which are coupled to the first and second inputs of the first amplifier.

At 920, the first amplifier can supply a controlled current first electrostimulation output waveform through a first patient electrode terminal.

At 930, an output of the first amplifier can be supplied to second amplifier and received as a control signal thereof.

At 940, the second amplifier can supply a controlled voltage second electrostimulation output waveform via a second patient electrode terminal. In an example, the first and second electrostimulation output waveforms can be supplied via the first and second patient electrode terminals concurrently. The second electrostimulation output waveform can be substantially inverted in sign with respect to the first electrostimulation output waveform. In an example, concurrent delivery of each of the first and second electrostimulation output waveforms to the patient can facilitate charge-balanced electrostimulation of a patient target site. In an example, the technique can include translating the first and second electrostimulation output waveforms from amplifier power supply levels to electrostimulation power supply levels.

The technique can also involve modulating at least one parameter of the charge-balanced electrostimulation based on feedback indications related to changes in impedance at the electrode terminals, allowing the device to adapt to varying conditions at the electrode-skin interface in real-time.

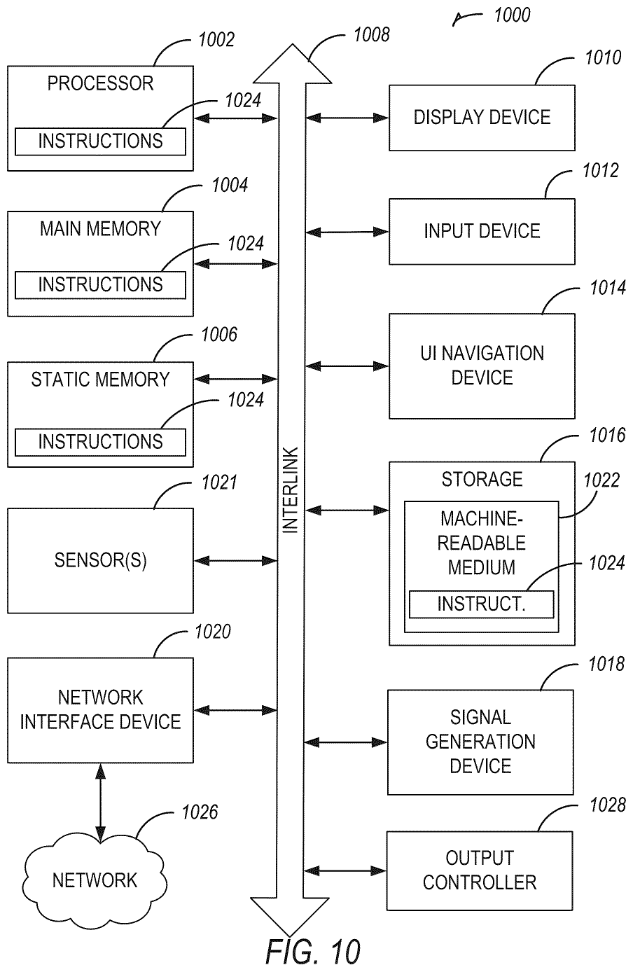

FIG. 10 illustrates generally an example of a block diagram of a machine 1000 upon which any one or more of the techniques (e.g., methodologies) discussed herein may perform in accordance with some examples. In alternative embodiments, the machine 1000 may operate as a standalone device or may be connected (e.g., networked) to other machines. In a networked deployment, the machine 1000 may operate in the capacity of a server machine, a client machine, or both in server-client network environments. In an example, the machine 1000 may act as a peer machine in peer-to-peer (P2P) (or other distributed) network environment. The machine 1000 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In an example, the hardware may include configurable execution units (e.g., transistors, circuits, etc.) and a computer readable medium containing instructions, where the instructions configure the execution units to carry out a specific operation when in operation. The configuring may occur under the direction of the executions units or a loading mechanism. Accordingly, the execution units are communicatively coupled to the computer readable medium when the device is operating. In this example, the execution units may be a member of more than one module. For example, under operation, the execution units may be configured by a first set of instructions to implement a first module at one point in time and reconfigured by a second set of instructions to implement a second module.

Machine (e.g., computer system) 1000 may include a hardware processor 1002 (e.g., a central processing unit (CPU), a graphics processing unit (GPU), a hardware processor core, or any combination thereof), a main memory 1004 and a static memory 1006, some or all of which may communicate with each other via an interlink (e.g., bus) 1008. The machine 1000 may further include a display unit 1010, an alphanumeric input device 1012 (e.g., a keyboard), and a user interface (UI) navigation device 1014 (e.g., a mouse). In an example, the display unit 1010, alphanumeric input device 1012 and UI navigation device 1014 may be a touch screen display. The machine 1000 may additionally include a storage device (e.g., drive unit) 1016, a signal generation device 1018 (e.g., a speaker), a network interface device 1020, and one or more sensors 1021, such as a global positioning system (GPS) sensor, compass, accelerometer, or other sensor. The machine 1000 may include an output controller 1028, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1016 may include a machine readable medium 1022 that is non-transitory on which is stored one or more sets of data structures or instructions 1024 (e.g., software) embodying or utilized by any one or more of the techniques or functions described herein. The instructions 1024 may also reside, completely or at least partially, within the main memory 1004, within static memory 1006, or within the hardware processor 1002 during execution thereof by the machine 1000. In an example, one or any combination of the hardware processor 1002, the main memory 1004, the static memory 1006, or the storage device 1016 may constitute machine readable media.

While the machine readable medium 1022 is illustrated as a single medium, the term “machine readable medium” may include a single medium or multiple media (e.g., a centralized or distributed database, or associated caches and servers) configured to store the one or more instructions 1024.

The term “machine readable medium” may include any medium that is capable of storing, encoding, or carrying instructions for execution by the machine 1000 and that cause the machine 1000 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1024 may further be transmitted or received over a communications network 1026 using a transmission medium via the network interface device 1020 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 1002.11 family of standards known as Wi-Fi®, IEEE 1002.16 family of standards known as WiMax®), IEEE 1002.15.4 family of standards, peer-to-peer (P2P) networks, among others. In an example, the network interface device 1020 may include one or more physical jacks (e.g., Ethernet, coaxial, or phone jacks) or one or more antennas to connect to the communications network 1026. In an example, the network interface device 1020 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1000, and includes digital or analog communications signals or other intangible medium to facilitate communication of such software.

The following, non-limiting examples, detail certain aspects of the present subject matter to solve the challenges and provide the benefits discussed herein, among others.

Example 1 is an electrostimulation electronics unit. This unit can include control waveform generation circuitry that provides an output for at least one alternating current (AC) first control waveform and a first amplifier that receives the AC first control waveform and supplies a controlled current first electrostimulation output waveform via a first patient electrode terminal. Additionally, the unit can include a second amplifier that receives an AC second control waveform and supplies a controlled voltage second electrostimulation output waveform via a second patient electrode terminal. Together, these output waveforms can provide charge-balanced electrostimulation to a patient over a full AC electrostimulation cycle.

Example 2 can include the subject matter of Example 1, with the additional feature that the first and second electrostimulation output waveforms are inverted in sign with respect to each other.

Example 3 can include the subject matter of any one of Examples 1 to 2, further comprising a power converter that provides a DC electrostimulation power supply. The first and second amplifiers can be connected to respective first and second boost circuits that translate the output waveforms from amplifier power supply levels to electrostimulation power supply levels.

Example 4 can include the subject matter of Example 3, wherein the output voltage of the power converter is adjusted based on an electrical load on the power converter.

Example 5 can include the subject matter of any one of Examples 3 to 4, with the additional feature that the at least one AC first control waveform includes a differential current signal of opposing polarity components that are respectively coupled to first and second inputs of the first amplifier.

Example 6 can include the subject matter of Example 5, wherein the first input of the first amplifier is coupled to the first boost circuit to receive a feedback indication of current through the first boost circuit in accordance with a change of impedance at the patient electrode terminals. The second input of the first amplifier can be coupled to the second boost circuit to receive a feedback indication of current through the second boost circuit in accordance with a change of impedance at the patient electrode terminals.

Example 7 can include the subject matter of Example 6, where the second input of the second amplifier is coupled to the second boost circuit to receive a feedback indication of voltage at the second patient electrode terminal.

Example 8 can include the subject matter of any one of Examples 6 to 7, wherein the first input of the second amplifier is coupled to a bias voltage from a soft-start ramp generator that adjusts the bias voltage in response to power-on.

Example 9 can include the subject matter of Example 8, wherein the first input of the first amplifier is coupled to receive a signal based on a difference between the bias voltage from the soft-start ramp generator and a feedback indication of voltage at the first patient electrode terminal.

Example 10 can include the subject matter of any one of Examples 3 to 9, wherein the first boost circuit has a higher output impedance than the second boost circuit.

Example 11 can include the subject matter of any one of Examples 1 to 10, wherein the first amplifier includes a first integration capacitor in a first feedback path between an output of the first amplifier and the first input of the first amplifier. The second amplifier includes a second integration capacitor in a second feedback path between an output of the second amplifier and the first input of the second amplifier.

Example 12 can include the subject matter of any one of Examples 1 to 11, comprising an arrangement of protection diodes, coupled to at least one of the patient electrode terminals and configured to limit a voltage at the patient electrode terminals with respect to a DC electrostimulation power supply voltage and a ground reference voltage.

Example 13 is a method for responding to rapid changes in load impedance at an electrode-skin interface. This method involves receiving at least one control waveform via a first amplifier, supplying a controlled current first electrostimulation output waveform via a first patient electrode terminal, receiving at least one control waveform via a second amplifier, and supplying a controlled voltage second electrostimulation output waveform via a second patient electrode terminal. The method also can include sensing a change in load impedance at the patient electrode terminals, receiving feedback indications of current, and modulating an output current of at least the first electrostimulation output waveform based on the feedback indications.

Example 14 is a method for providing transcutaneous electrostimulation for a restless legs syndrome (RLS) patient via an electrostimulation device. This method can include receiving an AC first control waveform via a first amplifier, supplying a controlled current first electrostimulation output waveform via a first patient electrode terminal, receiving an AC second control waveform via a second amplifier, and supplying a controlled voltage second electrostimulation output waveform via a second patient electrode terminal. The concurrent supply of the first and second electrostimulation output waveforms can provide charge balanced electrostimulation at a patient-electrode interface over a full AC electrostimulation cycle.

Example 15 can include the subject matter of Example 14, with the additional feature that the first and second electrostimulation output waveforms are inverted in sign with respect to each other.

Example 16 can include the subject matter of Example 14, further comprising translating the first and second electrostimulation output waveforms from amplifier power supply levels to electrostimulation power supply levels.

Example 17 can include the subject matter of Example 16, further comprising adjusting an output voltage of a power converter of the electrostimulation device based on sensed electrical load on the power converter.

Example 18 can include the subject matter of any one of Examples 16 to 17, wherein the AC first control waveform includes a differential current signal of opposing polarity components that are respectively coupled to first and second inputs of the first amplifier.

Example 19 can include the subject matter of Example 18, comprising receiving a first feedback indication of current in accordance with a change of impedance at the patient electrode terminals, receiving a second feedback indication of current in accordance with a change of impedance at the patient electrode terminals, and modulating at least one parameter of the charge balanced electrostimulation based on the received feedback indications.

Example 20 can include the subject matter of any one of Examples 14 to 19, comprising limiting a voltage at the patient electrode terminals, with respect to a DC electrostimulation power supply voltage and a ground reference voltage, via an arrangement of protection diodes, coupled to at least one of the patient electrode terminals.

Example 21 is a device for providing a desired current output of an electrostimulation waveform to an external target body location via an electrostimulation electrode placed at the external target body location. The device can include impedance detection circuitry configured to sense a first indication of a first impedance and a second indication of a second impedance at the external target body location. It also can include an electrostimulation electronics unit configured to generate a high-frequency (HF) electrostimulation waveform for delivery to the external target body location in accordance with the sensed first impedance. The waveform modulation circuitry can be configured to regulate adjustment of at least one parameter of the HF electrostimulation waveform to adjust the current toward a specified first current. Additionally, voltage clamp componentry can be configured to limit an overvoltage at the external target body location during the adjustment of the waveform.

Example 22 can include the subject matter of Example 21, wherein the electrostimulation electronics unit includes a first electrostimulation waveform amplifier configured to receive an output waveform from the waveform modulation circuitry and output a controlled-current amplified waveform to a first electrode terminal. It also can include a second electrostimulation waveform amplifier configured to receive an output waveform from the first electrostimulation waveform amplifier and output a controlled-voltage amplified waveform to a second electrode terminal.

Example 23 can include the subject matter of Example 22, wherein the impedance detection circuitry is configured to sense an output current of at least one of the first and second electrostimulation waveform amplifiers and attenuate an output current of the first electrostimulation waveform amplifier when the sensed output current exceeds a specified threshold.

Example 24 can include the subject matter of Example 21, comprising a charge-delivery interface, configured to couple with the electrostimulation electrode. The charge-delivery interface can be configured to sense a change in pressure applied thereto corresponding with a sensed second indication of the second impedance.

Example 25 can include the subject matter of Example 24, wherein the charge-delivering interface is configured to sense intermittent skin contact between the charge-delivering interface and the external target body location.

Example 26 can include the subject matter of any one of Examples 21 to 25, comprising at least one leg sensor configured to sense rapid leg movement therapy of a subject corresponding with the second indication of the second impedance.

Example 27 can include the subject matter of any one of Examples 21 to 26, wherein the voltage clamp componentry is configured to control a maximum overcurrent peak to remain less than 300% of the specified first current before adjusting the current toward the specified first current.

Example 28 can include the subject matter of Example 27, wherein the waveform modulation circuitry is configured to adjust the current toward the specified first current in less than 35 microseconds (μs).

Example 29 can include the subject matter of any one of Examples 21 to 28, wherein the voltage clamp componentry is configured to control a maximum overcurrent peak to remain less than 600% of the specified first current before adjusting the current toward the specified first current.

Example 30 can include the subject matter of Example 29, wherein the waveform modulation circuitry is configured to regulate adjustment of the current toward the specified first current in less than 5 μs.

Example 31 can include the subject matter of any one of Examples 21 to 30, wherein the voltage clamp componentry is configured to limit a maximum overcurrent peak, resulting from the change between the first impedance and the second impedance, such that the peak transfers less than 800 nanocoulombs (nC) of charge to the external target body location before the current of the electrostimulation waveform is reverted to the specified first current.

Example 32 can include the subject matter of any one of Examples 21 to 31, wherein the waveform modulation circuitry is configured to trigger adjusting at least one parameter of the HF electrostimulation waveform in response to the second indication of changing from the first indication of the first impedance by at least a specified threshold value, wherein the specified threshold value is less than ±20 ohms (Ω).

Example 33 can include the subject matter of any one of Examples 21 to 32, wherein the waveform modulation circuitry is configured to trigger adjusting at least one parameter of the HF electrostimulation waveform in response to the first indication of the first impedance being within a range of 300Ω and 600Ω and the second indication of the second impedance being at a value indicating an open circuit.

Example 34 can include the subject matter of any one of Examples 21 to 33, wherein the waveform modulation circuitry is configured to trigger adjusting at least one parameter of the HF electrostimulation waveform in response to the first indication of the first impedance being at a value indicating an open circuit and the second indication of the second impedance being within a range of 300Ω and 600Ω.

Example 35 can include the subject matter of any one of Examples 21 to 34, wherein the waveform modulation circuitry is configured to trigger adjusting at least one parameter of the HF electrostimulation waveform in response to the first indication of the first impedance being within a range of 300Ω and 600Ω and the second indication of the second impedance being at a value indicating a short circuit.

Example 36 can include the subject matter of any one of Examples 21 to 35, wherein the waveform modulation circuitry is configured to trigger adjusting at least one parameter of the HF electrostimulation waveform in response to the first indication of the first impedance being at a value indicating a short circuit and the second indication of the second impedance being within a range of 300Ω and 600Ω.

Example 37 can include the subject matter of any one of Examples 21 to 36, wherein the waveform modulation circuitry is configured to trigger adjusting at least one parameter of the HF electrostimulation waveform in response to the first indication of the first impedance being within a range of 900Ω and 1.1 kiloohms (kΩ) and the second indication of the second impedance being within a range of 90Ω and 110Ω.

Example 38 can include the subject matter of any one of Examples 21 to 37, wherein the waveform modulation circuitry is configured to trigger adjusting at least one parameter of the HF electrostimulation waveform in response to the first indication of the first impedance being within a range of 90Ω and 110Ω and the second indication of the second impedance being within a range of 900Ω and 1.1 kΩ.

Example 39 is a method for providing a desired current output of an electrostimulation waveform. This method can include sensing a first indication of a first impedance using an electrostimulation electrode at an external target body location, delivering a high-frequency (HF) electrostimulation waveform to the external target body location in accordance with the sensed first impedance, sensing a second indication of a second impedance, adjusting at least one parameter of the HF electrostimulation waveform to adjust the current toward a specified first current, and limiting an accumulation of charge at the external target body location during the adjustment of the waveform.

Example 40 can include the subject matter of Example 39, wherein sensing the second indication of the second impedance includes sensing a change in pressure applied to a charge-delivering interface.

Example 41 can include the subject matter of any one of Examples 39 to 40, wherein sensing the second indication of the second impedance includes sensing intermittent skin contact between a charge delivering interface and the external target body location.

Example 42 can include the subject matter of any one of Examples 39 to 41, wherein sensing the second indication of the second impedance includes sensing rapid leg movement therapy of a subject.

Example 43 can include the subject matter of any one of Examples 39 to 42, wherein limiting an accumulation of charge at the external target body location includes controlling a maximum overcurrent peak to remain less than 300% of the specified first current before adjusting the current toward the specified first current.

Example 44 can include the subject matter of Example 43, wherein adjusting at least one parameter of the HF electrostimulation waveform includes adjusting the current toward the specified first current in less than 35 microseconds (μs).

Example 45 can include the subject matter of any one of Examples 39 to 44, wherein limiting an accumulation of charge at the external target body location includes controlling a maximum overcurrent peak to remain less than 600% of the specified first current before adjusting the current toward the specified first current.

Example 46 can include the subject matter of Example 45, wherein adjusting at least one parameter of the HF electrostimulation waveform includes adjusting the current toward the specified first current in less than 5 μs.

Example 47 can include the subject matter of any one of Examples 39 to 46, wherein limiting an accumulation of charge at the external target body location includes governing the waveform such that maximum overcurrent peaking, resulting from the change between the first impedance and the second impedance, transfers less than 800 nanocoulombs (nC) of charge to the external target body location before the current of the electrostimulation waveform is reverted to the specified first current.

Example 48 can include the subject matter of any one of Examples 39 to 47, comprising triggering adjusting at least one parameter of the HF electrostimulation waveform in response to the second indication of changing from the first indication of the first impedance by at least a specified threshold value, wherein the specified threshold value is less than ±20 ohms (Ω).

Example 49 can include the subject matter of any one of Examples 39 to 48, comprising triggering adjusting at least one parameter of the HF electrostimulation waveform in response to the first indication of the first impedance being within a range of 300Ω and 600Ω and the second indication of the second impedance being at a value indicating an open circuit.

Example 50 can include the subject matter of any one of Examples 39 to 49, comprising triggering adjusting at least one parameter of the HF electrostimulation waveform in response to the first indication of the first impedance being at a value indicating an open circuit and the second indication of the second impedance being within a range of 300Ω and 600Ω.

Example 51 can include the subject matter of any one of Examples 39 to 50, comprising triggering adjusting at least one parameter of the HF electrostimulation waveform in response to the first indication of the first impedance being within a range of 300Ω and 600Ω and the second indication of the second impedance being at a value indicating a short circuit.

Example 52 can include the subject matter of any one of Examples 39 to 51, comprising triggering adjusting at least one parameter of the HF electrostimulation waveform in response to the first indication of the first impedance being at a value indicating a short circuit and the second indication of the second impedance being within a range of 300Ω and 600Ω.

Example 53 can include the subject matter of any one of Examples 39 to 52, comprising triggering adjusting at least one parameter of the HF electrostimulation waveform in response to the first indication of the first impedance being within a range of 900Ω and 1.1 kiloohms (kΩ) and the second indication of the second impedance being within a range of 90Ω and 110Ω.

Example 54 can include the subject matter of any one of Examples 39 to 53, comprising triggering adjusting at least one parameter of the HF electrostimulation waveform in response to the first indication of the first impedance being within a range of 90Ω and 110Ω and the second indication of the second impedance being within a range of 900Ω and 1.1 kΩ.

Example 55 is a transcutaneous neurostimulation therapy device for treating a patient with symptoms associated with Restless Legs Syndrome (RLS) or Periodic Limb Movement Disorder (PLMD) using applied high-frequency electrostimulation. The device can include an electrostimulation electronics unit with first and second skin electrodes coupled to waveform generation circuitry for delivering a bi-phasic pulsed, charge-balanced therapy waveform to the patient at an external target body location. The waveform can be delivered at a frequency between 500 hertz (Hz) and 10,000 Hz and, when delivered at 500 ohms (Ω) of resistance, applies an output voltage between 18 Volts (V) and 22 V, an output current between 35 milliamps (mA) and 45 mA, and an average power density between 0.03 Watts per Square Centimeter (W/cm²) and 0.06 W/cm².

Example 56 can include the subject matter of Example 55, wherein when the first and second skin electrodes are applied at the external target body location at 500Ω of resistance, the electrostimulation electronics unit is operable to establish or adjust the therapy waveform to provide a maximum output voltage between 19.5 V and 20.5 V.

Example 57 can include the subject matter of any one of Examples 55 to 56, wherein when the first and second skin electrodes are applied at the external target body location at 500Ω of resistance, the electrostimulation electronics unit is operable to establish or adjust the therapy waveform to provide a maximum output current between 38 mA and 42 mA.

Example 58 can include the subject matter of any one of Examples 55 to 57, wherein when the first and second skin electrodes are applied at the external target body location at 500 ohms of resistance, the electrostimulation electronics unit is operable to establish or adjust the therapy waveform to provide a maximum average power density between 0.045 W/cm² and 0.05 W/cm².

Example 59 can include the subject matter of any one of Examples 55 to 58, wherein the waveform is delivered to include a pulse at a pulse duration between 200 microseconds (μs) and 300 μs.

Example 60 can include the subject matter of any one of Examples 55 to 59, wherein the waveform is delivered at a maximum pulse charge between 4 microcoulombs (μC) and 65 μC.

Example 61 can include the subject matter of any one of Examples 55 to 60, wherein when the first and second skin electrodes are applied at the external target body location at 500Ω of resistance, the electrostimulation electronics unit is operable to establish or adjust the therapy waveform to provide a maximum current density between 2.2 milliAmps per square centimeter (mA/cm²) and 2.6 mA/cm².

Example 62 can include the subject matter of any one of Examples 55 to 61, wherein the therapy waveform is a multiphasic waveform including symmetrical anodic and cathodic phases with respective durations between 120 μs and 125 μs.

Example 63 is a transcutaneous neurostimulation therapy device for treating a patient with symptoms associated with Restless Legs Syndrome (RLS) or Periodic Limb Movement Disorder (PLMD) using applied high-frequency electrostimulation. The device comprises electrostimulation waveform generation circuitry for generating a bi-phasic pulsed, charge-balanced therapy waveform at a frequency between 500 hertz (Hz) and 10,000 Hz. The waveform is for delivery to the patient at an external target body location via electrostimulation electrodes couplable to the electrostimulation waveform generation circuitry. The device also can include electrostimulation waveform regulator circuitry for regulating the therapy waveform such that upon encountering a resistance of 500 ohms (Ω) at the electrostimulation electrodes, the waveform is regulated at an output voltage between 18 Volts (V) and 22 V, an output current of the therapy waveform between 35 milliamps (mA) and 45 mA, and an average power density of the therapy waveform between 0.03 Watts per Square Centimeter (W/cm²) and 0.06 W/cm².

Example 64 can include the subject matter of Example 63, wherein when the electrostimulation electrodes are applied at the external target body location at 500Ω of resistance, the electrostimulation waveform generation circuitry is operable to establish or adjust the therapy waveform to provide a maximum output voltage between 19.5 V and 20.5 V.

Example 65 can include the subject matter of any one of Examples 63 to 64, wherein when the electrostimulation electrodes are applied at the external target body location at 500Ω of resistance, the electrostimulation waveform generation circuitry is operable to establish or adjust the therapy waveform to provide a maximum output current between 38 mA and 42 mA.

Example 66 can include the subject matter of any one of Examples 63 to 65, wherein when the electrostimulation electrodes are applied at the external target body location at 500 ohms of resistance, the electrostimulation waveform generation circuitry is operable to establish or adjust the therapy waveform to provide a maximum average power density between 0.045 W/cm² and 0.05 W/cm².

Example 67 can include the subject matter of any one of Examples 63 to 66, wherein the waveform is delivered to include a pulse at a pulse duration between 200 microseconds (μs) and 300 μs.

Example 68 can include the subject matter of any one of Examples 63 to 67, wherein the waveform is delivered at a maximum pulse charge between 4 microcoulombs (μC) and 65μC.

Example 69 is a method for providing a desired high-frequency electrostimulation waveform. This method can include generating, via an electrostimulation electronics unit, a bi-phasic pulsed, charge-balanced therapy waveform at a frequency between 500 hertz (Hz) and 10,000 Hz. It also involves regulating, via electrostimulation waveform regulator circuitry, an output voltage of the therapy waveform at between 18 Volts (V) and 22 V, an output current at between 35 milliamps (mA) and 45 mA, and an average power density at between 0.03 Watts per Square Centimeter (W/cm²) and 0.06 W/cm² when the therapy waveform is delivered, via electrostimulation electrodes, at a resistance of 500 ohms (Ω).

Example 70 can include the subject matter of Example 69, wherein regulating the therapy waveform includes establishing or adjusting a maximum output voltage to a value between 19.5 V and 20.5 V when the therapy waveform is delivered, via the electrostimulation electrodes, at a resistance of 500 ohms (Ω).

Example 71 can include the subject matter of any one of Examples 69 to 70, wherein regulating the therapy waveform includes establishing or adjusting a maximum output current to a value between 38 mA and 42 mA when the therapy waveform is delivered, via the electrostimulation electrodes, at a resistance of 500 ohms (Ω).

Example 72 can include the subject matter of any one of Examples 69 to 71, wherein regulating the therapy waveform includes establishing or adjusting a maximum average power density to a value between 0.045 W/cm² and 0.05 W/cm² when the therapy waveform is delivered, via the electrostimulation electrodes, at a resistance of 500 ohms (Ω).

Example 73 can include the subject matter of any one of Examples 69 to 72, wherein generating the bi-phasic pulsed, charge-balanced therapy waveform includes generating waveform pulses at a pulse duration between 200 microseconds (μs) and 300 μs.

Example 74 can include the subject matter of any one of Examples 69 to 73, wherein generating the bi-phasic pulsed, charge-balanced therapy waveform includes generating waveform at a maximum pulse charge between 4 microcoulombs (μC) and 65 μC.

Example 75 can include the subject matter of any one of Examples 69 to 74, wherein regulating the therapy waveform includes establishing or adjusting a maximum current density between 2.2 milliAmps per square centimeter (mA/cm²) and 2.6 mA/cm² when the therapy waveform is delivered, via the electrostimulation electrodes, at a resistance of 500 ohms (Ω).

Example 76 can include the subject matter of any one of Examples 69 to 75, wherein the therapy waveform is a multiphasic waveform including symmetrical anodic and cathodic phases with respective durations between 120 μs and 125 μs.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A transcutaneous neurostimulation therapy device for treating a patient having one or more symptoms associated with at least one of Restless Legs Syndrome (RLS) or Periodic Limb Movement Disorder (PLMD) using applied high-frequency electrostimulation, the device comprising:
an electrostimulation electronics unit including first and second skin electrodes coupled to waveform generation circuitry for delivering a bi-phasic pulsed, charge-balanced therapy waveform to the patient at an external target body location, the waveform delivered at a frequency of between 500 hertz (Hz) and 10,000 Hz;
wherein when delivered to the external target body location at 500 ohms (Ω) of resistance, the therapy waveform applies an output voltage of between 18 Volts (V) and 22 V, an output current between 35 milliamps (mA) and 45 mA, and an average power density between 0.03 Watts per Square Centimeter (W/cm2) and 0.06 W/cm2.

2. The device of claim 1, wherein when the first and second skin electrodes are applied at the external target body location at 500Ω of resistance, the electrostimulation electronics unit is operable to establish or adjust the therapy waveform to provide a maximum output voltage between 19.5 V and 20.5 V.

3. The device of claim 1, wherein when the first and second skin electrodes are applied at the external target body location at 500Ω of resistance, the electrostimulation electronics unit is operable to establish or adjust the therapy waveform to provide a maximum output current between 38 mA and 42 mA.

4. The device of claim 1, wherein when the first and second skin electrodes are applied at the external target body location at 500 ohms of resistance, the electrostimulation electronics unit is operable to establish or adjust the therapy waveform to provide a maximum average power density between 0.045 W/cm2 and 0.05 W/cm2.

5. The device of claim 1, wherein the waveform is delivered to include a pulse at a pulse duration between 200 microseconds (μs) and 300 μs.

6. The device of claim 1, wherein the waveform is delivered at a maximum pulse charge between 4 microcoulombs (μC) and 65 μC.

7. The device of claim 1, wherein when the first and second skin electrodes are applied at the external target body location at 500Ω of resistance, the electrostimulation electronics unit is operable to establish or adjust the therapy waveform to provide a maximum current density between 2.2 milliAmps per square centimeter (mA/cm2) and 2.6 mA/cm2.

8. The device of claim 1, wherein the therapy waveform is a multiphasic waveform including symmetrical anodic and cathodic phases with respective durations between 120 μs and 125 μs.

9. A transcutaneous neurostimulation therapy device for treating a patient having one or more symptoms associated with at least one of Restless Legs Syndrome (RLS) or Periodic Limb Movement Disorder (PLMD) using applied high-frequency electrostimulation, the device comprising:
electrostimulation waveform generation circuitry for generating a bi-phasic pulsed, charge-balanced therapy waveform at a frequency of between 500 hertz (Hz) and 10,000 Hz, the waveform for delivery to the patient at an external target body location via electrostimulation electrodes couplable to the electrostimulation waveform generation circuitry; and
electrostimulation waveform regulator circuitry for regulating the therapy waveform such that upon encountering a resistance of ohms (Ω) at the electrostimulation electrodes, the waveform is regulated at an output voltage between 18 Volts (V) and 22 V, an output current of the therapy waveform between 35 milliamps (mA) and 45 mA, and an average power density of the therapy waveform between 0.03 Watts per Square Centimeter (W/cm2) and 0.06 W/cm2.

10. The device of claim 9, wherein when the electrostimulation electrodes are applied at the external target body location at 500Ω of resistance, the electrostimulation waveform generation circuitry is operable to establish or adjust the therapy waveform to provide a maximum output voltage between 19.5 V and 20.5 V.

11. The device of claim 9, wherein when the electrostimulation electrodes are applied at the external target body location at 500Ω of resistance, the electrostimulation waveform generation circuitry is operable to establish or adjust the therapy waveform to provide a maximum output current between 38 mA and 42 mA.

12. The device of claim 9, wherein when the electrostimulation electrodes are applied at the external target body location at 500 ohms of resistance, the electrostimulation waveform generation circuitry is operable to establish or adjust the therapy waveform to provide a maximum average power density between 0.045 W/cm2 and 0.05 W/cm2.

13. The device of claim 9, wherein the waveform is delivered to include a pulse at a pulse duration between 200 microseconds (μs) and 300 μs.

14. The device of claim 9, wherein the waveform is delivered at a maximum pulse charge between 4 microcoulombs (μC) and 65 μC.

15. A method for providing a desired high-frequency electrostimulation waveform, the method comprising:
generating, via an electrostimulation electronics unit, a bi-phasic pulsed, charge-balanced therapy waveform at a frequency of between 500 hertz (Hz) and 10,000 Hz; and
regulating, via electrostimulation waveform regulator circuitry, an output voltage of the therapy waveform at between 18 Volts (V) and 22 V, an output current at between 35 milliamps (mA) and 45 mA, and an average power density at between 0.03 Watts per Square Centimeter (W/cm2) and 0.06 W/cm2 when the therapy waveform is delivered, via electrostimulation electrodes, at a resistance of 500 ohms (Ω).

16. The method of claim 15, wherein regulating the therapy waveform includes establishing or adjusting a maximum output voltage to a value between 19.5 V and 20.5 V when the therapy waveform is delivered, via the electrostimulation electrodes, at a resistance of 500 ohms (Ω).

17. The method of claim 15, wherein regulating the therapy waveform includes establishing or adjusting a maximum output current to a value between 38 mA and 42 mA when the therapy waveform is delivered, via the electrostimulation electrodes, at a resistance of 500 ohms (Ω).

18. The method of claim 15, wherein regulating the therapy waveform includes establishing or adjusting a maximum average power density to a value between 0.045 W/cm2 and 0.05 W/cm2 when the therapy waveform is delivered, via the electrostimulation electrodes, at a resistance of 500 ohms (Ω).

19. The method of claim 15, wherein generating the bi-phasic pulsed, charge-balanced therapy waveform includes generating waveform pulses at a pulse duration between 200 microseconds (μs) and 300 μs.

20. The method of claim 15, wherein generating the bi-phasic pulsed, charge-balanced therapy waveform includes generating waveform at a maximum pulse charge between 4 microcoulombs (μC) and 65 μC.
